(19) United States
(12) Patent Application Publication
(10) Pub. No.: US 2026/0191543 A1
(43) Pub. Date: Jul. 9, 2026

(54) DISTAL FEMORAL JIG INSTRUMENT AND METHOD OF USING THE SAME IN AN ORTHOPAEDIC SURGICAL KNEE PROCEDURE

(71) Applicant: DePuy Ireland Unlimited Company, Ringaskiddy (IE)

(72) Inventors: Dustin N. Albert, Warsaw, IN (US); Jason M. Chavarria, Warsaw, IN (US); Jeremiah M. Lewis, Warsaw, IN (US); Nicholas A. Miltner, Fort Wayne, IN (US)

(21) Appl. No.: 19/417,901

(22) Filed: Dec. 12, 2025

Related U.S. Application Data
(60) Provisional application No. 63/741,619, filed on Jan. 3, 2025.

(51) Int. Cl. A61B 17/17

(52) U.S. Cl. CPC A61B 17/1764

(57) ABSTRACT

A distal femoral jig includes an alignment guide and an outrigger component. A segmented intramedullary rod instrument may be used to install the alignment guide on a patient's femur. The alignment guide is configured to contact the distal femoral condyles of the patient's femur so as to align a distal cutting block coupled to the outrigger component. The distal femoral jig may be operated in either a mechanical alignment mode of operation or a kinematic alignment mode of operation. Methods of surgically preparing a patient's femur are also disclosed.

This application claims priority to U.S. Provisional Patent Application Ser. No. 63/741,619 which was filed on Jan. 3, 2025, which is hereby incorporated by reference in its entirety.

### CROSS REFERENCE

Cross reference is made to copending U.S. patent application Ser. No. ______ entitled “INTRAMEDULLARY ROD INSTRUMENT AND METHOD OF USING THE SAME IN AN ORTHOPAEDIC SURGICAL KNEE PROCEDURE” (Attorney Docket No. 265280-431226, DEP7189USNP2), which is assigned to the same assignee as the present application, filed concurrently herewith, and hereby incorporated by reference in its entirety.

### TECHNICAL FIELD

The present disclosure relates generally to orthopaedic surgical instruments and, more particularly, to surgical instruments used to resect a patient's femur.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

In total knee arthroplasty (TKA), the femur and tibia of the patient's knee are resected to create planar surfaces onto which a prosthetic femoral component and tibial component, respectively, are installed. Traditional TKA involves determining the resection planes based on a pre-determined angle as a function of mechanical alignment or by using a balanced approach that sets the resection planes based on ligament tension. More recently, kinematic alignment techniques involve determining the resection planes as a function of the native, pre-disease state of the patient's knee.

### SUMMARY

According to one aspect of the disclosure, an orthopaedic surgical instrument assembly for resecting distal condyles of a patient's femur during an orthopaedic knee replacement procedure includes a distal femoral cutting block having a mediolaterally extending cutting slot and an outrigger component having a block connector on its distal end. The distal femoral cutting block is secured to the outrigger component with the block connector. The instrument assembly also includes a hollow body component secured to the outrigger component. The body component has a bone facing surface formed in its distal end. The bone facing surface is configured to abut the distal condyles of the patient's femur. An indexing plate is secured to a proximal end of the body component. The indexing plate has a plurality of locking notches radially positioned around a proximal end thereof. The instrument assembly also includes an elongated, hollow rod housing positioned in the body component. A distal end of the rod housing is pivotally coupled to the distal end of the body component. The rod housing has an elongated bore extending therethrough that is configured to receive an intramedullary rod instrument. Further, the instrument assembly includes a locking collar slidably positioned on a proximal end of the rod housing. The locking collar has a locking tab distally extending away therefrom. The locking collar is positionable between a locked position in which the locking tab is positioned in one of the locking notches of the indexing plate so as to lock the rotational position of the rod housing relative to the body component, and a released position in which the locking tab is spaced apart from the locking notches of the indexing plate so as to allow the body component and the rod housing to freely rotate relative to one another.

In an embodiment, a cap component is positioned on the proximal end of the rod housing. The locking collar is positioned between the indexing plate and the cap component. The locking collar abuts the cap component when the locking collar is positioned in its released position, whereas the locking collar is spaced distally apart from the cap component when the locking collar is positioned in its locked position.

In an embodiment, a retaining lever is rotatably positioned on the proximal end of the rod housing. The retaining lever is positionable between a retained position in which the retaining lever retains the locking collar in its released position, and an unretained position in which the locking collar is positioned in its locked position.

A spring may be positioned on the proximal end of the rod housing. The spring biases the locking collar toward the indexing plate.

In an embodiment, a longitudinal axis of the elongated bore of the rod housing and a longitudinal axis of the cutting slot of the distal femoral cutting block define a cutting angle. Rotational movement of the body component and the rod housing relative to one another alters the cutting angle.

In an embodiment, each of the plurality of locking notches of the indexing plate corresponds to a predetermined cutting angle.

In an embodiment, a first grip is coupled to the locking collar and a second grip is coupled to the proximal end of the of the rod housing. The first grip is moveable relative to the second grip to cause the locking collar to move between its locked position and its released position.

In an embodiment, the locking collar includes a first slot and a second slot formed therein. The first slot extends generally parallel to a longitudinal axis of the elongated bore of the rod housing and the second slot extends orthogonally to the longitudinal axis of the elongated bore of the rod housing.

A retaining lever is rotatably positioned on the proximal end of the rod housing. The retaining lever is positionable between (i) a retained position in which the retaining lever is positioned in the second slot to retain the locking collar in its released position, and (ii) an unretained position in which the retaining lever is positioned in the first slot to position the locking collar in its locked position.

According to another aspect, a method of surgically preparing a patient's femur during an orthopaedic surgical knee procedure includes securing a distal femoral cutting block to a hollow body component of a distal femoral jig. The method also includes selecting between a kinematic alignment mode of operation of the distal femoral jig and a mechanical alignment mode of operation of the distal femoral jig. If the mechanical alignment mode of operation was selected, a locking collar of the distal femoral jig is positioned in a locked position so as to lock the distal femoral cutting block into one of a plurality of predetermined rotational positions. If the kinematic mode of operation was selected, the locking collar of the distal femoral jig is positioned in a released position so as to allow the distal femoral cutting block to rotate freely.

The distal femoral cutting block may be secured to an outrigger component of the distal femoral jig, with the outrigger component being secured to the hollow body component.

In an embodiment, the distal femoral jig includes a hollow rod housing positioned in, and pivotally coupled to, the hollow body component. The hollow body component has an indexing plate secured to a proximal end thereof, with the indexing plate having a plurality of locking notches. The locking collar is positioned on the rod housing and includes a locking tab. In such an embodiment, the locking tab of the locking collar is positioned in one of the plurality of locking notches of the indexing plate so as to lock the rotational position of the rod housing relative to the body component. In such an embodiment, the locking tab of the locking collar is retained spaced apart from the plurality of locking notches of the indexing plate so as to allow the body component and the rod housing to rotate freely relative to one another.

The locking tab of the locking collar may be retained spaced apart from the plurality of locking notches of the indexing plate with a retaining lever.

In an embodiment, the distal femoral jig includes a hollow rod housing positioned in, and pivotally coupled to, the hollow body component. In such an embodiment, the hollow body component of the distal femoral jig is positioned such that a bone facing surface of the hollow body component abuts distal condyles of the patient's femur and an intramedullary rod instrument is advanced through the hollow rod housing and into a reamed intramedullary canal of the patient's femur.

According to another aspect, a method of surgically preparing a patient's femur during an orthopaedic surgical knee procedure includes securing a distal femoral cutting block to a distal femoral jig, and advancing an intramedullary rod instrument through the distal femoral jig. The method also includes selecting between a kinematic alignment mode of operation of the distal femoral jig and a mechanical alignment mode of operation of the distal femoral jig. If the mechanical alignment mode of operation was selected, a locking collar of the distal femoral jig is positioned in a locked position so as to lock the distal femoral cutting block into one of a plurality of predetermined rotational positions. If the kinematic mode of operation was selected, the locking collar of the distal femoral jig is positioned in a released position so as to allow the distal femoral cutting block to rotate freely.

In an embodiment, the distal femoral jig includes a hollow rod housing positioned in, and pivotally coupled to, the hollow body component. The hollow body component has an indexing plate secured to a proximal end thereof, with the indexing plate having a plurality of locking notches. The locking collar is positioned on the rod housing and includes a locking tab. In such an embodiment, the locking tab of the locking collar is positioned in one of the plurality of locking notches of the indexing plate so as to lock the rotational position of the rod housing relative to the body component. In such an embodiment, the locking tab of the locking collar is retained spaced apart from the plurality of locking notches of the indexing plate so as to allow the body component and the rod housing to rotate freely relative to one another.

The locking tab of the locking collar may be retained spaced apart from the plurality of locking notches of the indexing plate with a retaining lever.

According to another aspect, a method of operating an orthopaedic surgical instrument includes securing a distal femoral cutting block to a hollow body component of a distal femoral jig and selecting between a kinematic alignment mode of operation of the distal femoral jig and a mechanical alignment mode of operation of the distal femoral jig. If the mechanical alignment mode of operation was selected, a locking collar of the distal femoral jig is positioned in a locked position so as to lock the distal femoral cutting block into one of a plurality of predetermined rotational positions relative to the a bone facing surface of the hollow body component, whereas if the kinematic mode of operation was selected, the locking collar of the distal femoral jig is positioned in a released position so as to allow the distal femoral cutting block to rotate freely relative to a bone facing surface of the hollow body component.

The distal femoral cutting block may be secured to an outrigger component of the distal femoral jig, with the outrigger component being secured to the hollow body component.

In an embodiment, the distal femoral jig includes a hollow rod housing positioned in, and pivotally coupled to, the hollow body component. The hollow body component has an indexing plate secured to a proximal end thereof, with the indexing plate having a plurality of locking notches. The locking collar is positioned on the rod housing and includes a locking tab. In such an embodiment, the locking tab of the locking collar is positioned in one of the plurality of locking notches of the indexing plate so as to lock the rotational position of the rod housing relative to the body component. In such an embodiment, the locking tab of the locking collar is retained spaced apart from the plurality of locking notches of the indexing plate so as to allow the body component and the rod housing to rotate freely relative to one another.

The locking tab of the locking collar may be retained spaced apart from the plurality of locking notches of the indexing plate with a retaining lever.

In an example, the distal femoral jig includes a hollow rod housing positioned in, and pivotally coupled to, the hollow body component, and the method further includes advancing an intramedullary rod instrument through the hollow rod housing.

According to one aspect of the disclosure, an orthopaedic surgical instrument for use in surgically preparing a patient's femur during an orthopaedic surgical knee procedure includes an intramedullary rod instrument configured to be advanced into a reamed intramedullary canal of the patient's femur. The intramedullary rod instrument includes a T-shaped handle and an elongated fluted rod extending distally away from the handle. The elongated fluted rod has a segmented proximal body and a non-segmented distal tip. The non-segmented distal tip has a maximum diameter. The segmented proximal body has a plurality of shaft segments formed therein, with each of the plurality of shaft segments including a main shaft section and a reduced shaft section. The main shaft section of each of the plurality of shaft segments has the same diameter as the maximum diameter of the non-segmented distal tip. The reduced shaft section of each of the plurality of shaft segments defines a reduced surface having a second diameter that is between 25-75% the size of the maximum diameter of the non-segmented distal tip. The reduced shaft section of each of the plurality of shaft segments has a superinferiorly extending fluid channel formed in each of its medial and lateral sides.

In an embodiment, the second diameter is approximately 66.6% the size of the maximum diameter of the non-segmented distal tip.

In an embodiment, each of the reduced shaft sections is secured to a main shaft section of an adjacent shaft segment.

The elongated fluted rod has an overall length, and the non-segmented distal tip has a length that may be between 20-25% of the overall length of the elongated fluted rod.

In an embodiment, the length of the non-segmented distal tip is approximately 20% of the overall length of the elongated fluted rod.

The elongated fluted rod may be constructed of metal.

In an embodiment, the non-segmented distal tip of the elongated fluted rod has a number of fluid channels formed therein, and the main shaft section of each of the plurality of shaft segments of the segmented proximal body of the elongated fluted rod also has a number of fluid channels formed therein. The fluid channels formed in the non-segmented distal tip are colinear with the fluid channels formed in the main shaft section of each of the plurality of shaft segments of the segmented proximal body.

In an embodiment, the non-segmented distal tip of the elongated fluted rod has a superinferiorly extending fluid channel formed in each of its anterior, posterior, medial, and lateral sides. The main shaft section of each of the plurality of shaft segments of the segmented proximal body of the elongated fluted rod has a superinferiorly extending fluid channel formed in each of its anterior, posterior, medial, and lateral sides.

In an example, the fluid channels formed in the non-segmented distal tip are colinear with the fluid channels formed in the main shaft section of each of the plurality of shaft segments of the segmented proximal body.

The fluid channels formed in the reduced shaft section of each of the plurality of shaft segments of the segmented proximal body of the elongated fluted rod are colinear with the fluid channels formed in the main shaft section of each of the plurality of shaft segments of the segmented proximal body.

According to another aspect, a method of surgically preparing a patient's femur during an orthopaedic surgical knee procedure includes securing a distal femoral cutting block to a distal femoral jig, and positioning the distal femoral cutting block such that a bone facing surface of the distal femoral jig abuts distal condyles of the patient's femur. The method includes advancing a non-segmented distal tip of an intramedullary rod instrument through the distal femoral jig and into a reamed intramedullary canal of the patient's femur. A segmented proximal body of the intramedullary rod instrument is advanced through the distal femoral jig and into the reamed intramedullary canal of the patient's femur such that the segmented proximal body of the intramedullary rod instrument flexes within a bowed section of the reamed intramedullary canal of the patient's femur. The segmented proximal body has a plurality of reduced shaft sections each of which defines a reduced surface having a diameter that is between 25-75% the size of a maximum diameter of the non-segmented distal tip. The plurality of reduced shaft sections also each define a superinferiorly extending fluid channel formed in each of its medial and lateral sides. The method also includes advancing a distal end of a T-shaped handle of the intramedullary rod instrument into contact with the distal femoral jig.

In an embodiment, a cutting slot of the distal femoral cutting block is aligned into a desired cutting angle with the distal femoral jig. The distal femoral cutting block may be pinned to the patient's femur with the cutting slot aligned at the desired cutting angle.

The intramedullary rod instrument is removed from the reamed intramedullary canal of the patient's femur subsequent to pinning of the distal femoral cutting block.

According to another aspect, an orthopaedic surgical instrument for use in surgically preparing a patient's femur during an orthopaedic surgical knee procedure includes an intramedullary rod instrument configured to be advanced into a reamed intramedullary canal of the patient's femur. The intramedullary rod instrument includes an anterposteriorly extending handle, and an elongated fluted rod extending superiorly away from the handle. The fluted rod includes a segmented proximal body and a non-segmented distal tip. The non-segmented distal tip has a maximum diameter. The segmented proximal body has a plurality of shaft segments formed therein, with each of the plurality of shaft segments including a main shaft section and a reduced shaft section. The main shaft section of each of the plurality of shaft segments has the same diameter as the maximum diameter of the non-segmented distal tip, with the reduced shaft section of each of the plurality of shaft segments having a second diameter that is less than the size of the maximum diameter of the non-segmented distal tip. The non-segmented distal tip of the fluted rod has a superinferiorly extending fluid channel formed in each of its anterior, posterior, medial, and lateral sides, with the main shaft section of each of the plurality of shaft segments of the segmented proximal body of the fluted rod also having a superinferiorly extending fluid channel formed in each of its anterior, posterior, medial, and lateral sides. The fluid channels formed in the non-segmented distal tip are colinear with the fluid channels formed in the main shaft section of each of the plurality of shaft segments of the segmented proximal body.

In an embodiment, the reduced shaft section of each of the plurality of shaft segments defines a reduced surface having a second diameter that is between 25-75% the size of the maximum diameter of the non-segmented distal tip.

The second diameter may be approximately 66.6% the size of the maximum diameter of the non-segmented distal tip.

In an embodiment, each of the reduced shaft sections is secured to a main shaft section of an adjacent shaft segment.

The elongated fluted rod has an overall length, and the non-segmented distal tip has a length that may be between 20-25% of the overall length of the elongated fluted rod. In a specific embodiment, the length of the non-segmented distal tip is approximately 20% of the overall length of the elongated fluted rod.

The elongated fluted rod may be constructed of metal.

### BRIEF DESCRIPTION OF THE DRAWINGS

The detailed description particularly refers to the following figures, in which:

FIG. 1 is a perspective view of a distal femoral jig for use in the surgical preparation of a patient's femur during performance of an orthopaedic knee procedure;

FIG. 2 is an enlarged fragmentary perspective view showing the locking collar of the distal femoral jig of FIG. 1 positioned in its locked position;

FIG. 3 is a view similar to FIG. 2, but showing the locking collar of the distal femoral jig of FIG. 1 positioned in its released position;

FIG. 4 is a plan view of the outrigger component and the alignment guide of the distal femoral jig of FIG. 1;

FIG. 5 is a perspective view of a segmented intramedullary rod instrument for use in the surgical preparation of a patient's femur during performance of an orthopaedic knee procedure;

FIG. 6 is an enlarged, fragmentary side view of the segmented intramedullary rod instrument of FIG. 5;

FIG. 7 is an enlarged cross sectional view of the segmented intramedullary rod instrument taken along the line 7-7 of FIG. 5, as viewed in the direction of the arrows;

FIG. 8 is a perspective view showing the distal femoral jig and the intramedullary rod instrument installed on the distal end of the patient's femur prior to performance of a distal resection of the patient's femur, note the distal femoral cutting block is secured to the outrigger component in FIG. 8;

FIG. 9 is a perspective view showing a distal femoral cutting block installed on the distal end of the patient's femur and being used during performance of a distal resection of the patient's femur;

FIG. 10 is a perspective view of another segmented intramedullary rod instrument for use in the surgical preparation of a patient's femur during performance of an orthopaedic knee procedure;

FIG. 11 is an enlarged, fragmentary side view of the segmented intramedullary rod instrument of FIG. 10;

FIG. 12 is a front perspective view of another distal femoral jig for use in the surgical preparation of a patient's femur during performance of an orthopaedic knee procedure; and

FIG. 13 is a rear perspective view of the distal femoral jig of FIG. 12.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

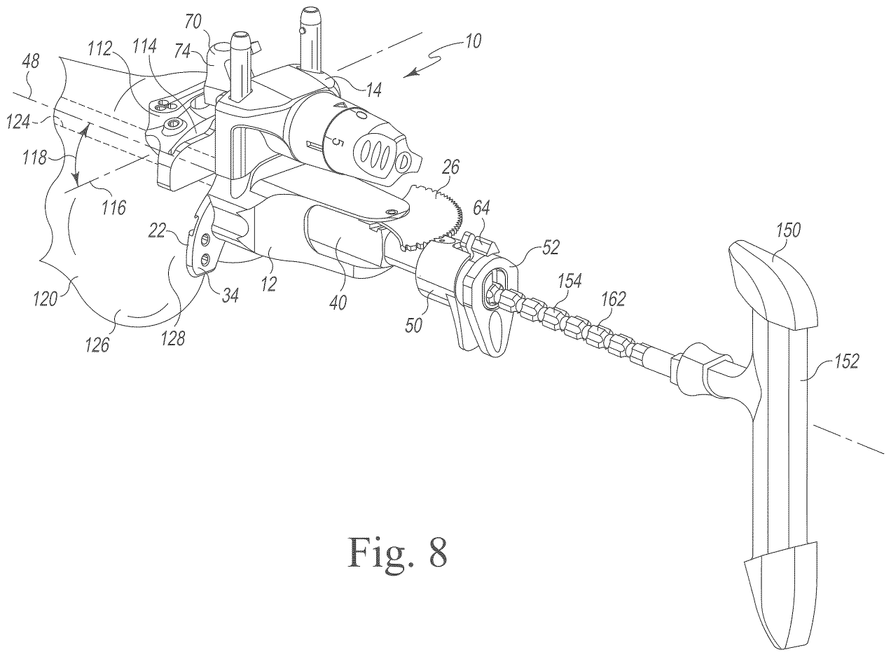

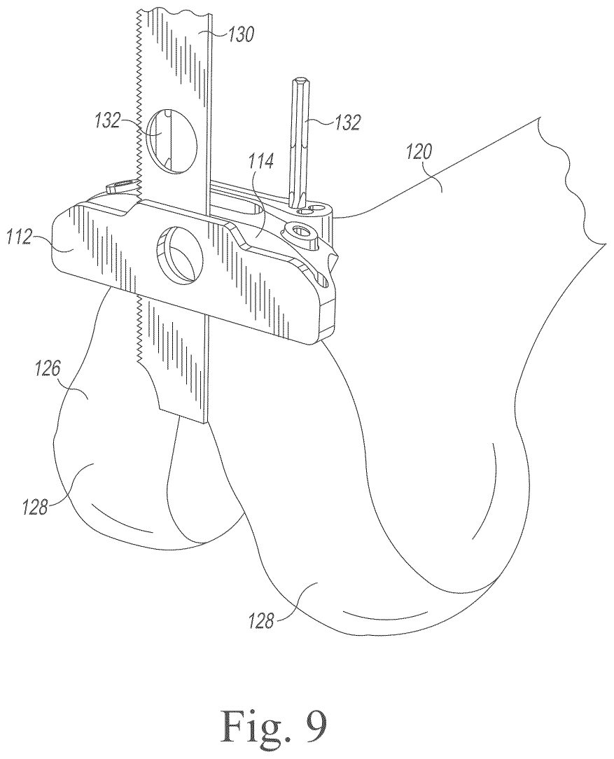

Referring to FIGS. 1-4, an orthopaedic surgical instrument 10—in the form of a distal femoral jig—for use in the surgical preparation of a patient's femur during performance of an orthopaedic knee procedure is shown. The distal femoral jig 10 includes an alignment guide 12 and a removable outrigger component 14. The distal femoral jig 10 is used to position and install a distal femoral cutting block 112 (see FIGS. 8 and 9) that is used to perform a distal cut on the patient's femur.

As can be seen in FIGS. 1-4, the alignment guide 12 of the distal femoral jig 10 includes a hollow body component 16 having a pair of anteriorly extending mounting posts 18. The outrigger component 14 includes a pair of mounting bores 20 which receive the mounting posts 18 to removably secure the outrigger component 14 to the body component 16. The body component 16 has a bone facing surface 22 formed in its distal end 24. As described below in greater detail, the bone facing surface 22 is configured to abut the distal condyles 128 of the patient's femur 120 during positioning and installation of the distal femoral cutting block 112.

An indexing plate 26 is secured to a proximal end 28 of the body component 16. The indexing plate 26 has a plurality of locking notches 30 radially positioned around its proximal end 32. Each of the plurality of locking notches 30 represents a desired varus/valgus angle (left or right—0 degrees to 14 degrees) for selection by a surgeon during use of the distal femoral jig 10.

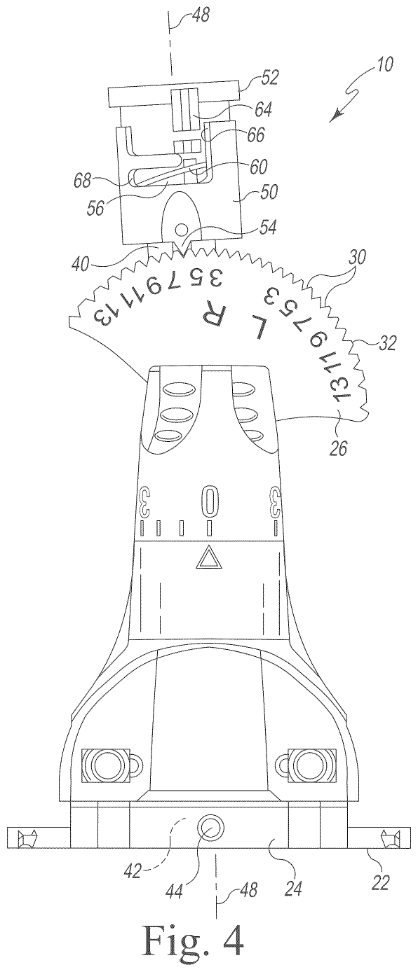

The alignment guide 12 also includes an elongated, hollow rod housing 40 positioned in the hollow body component 16. As can be seen in FIG. 4, a distal end 42 of the rod housing 40 is pivotally coupled to the distal end 24 of the body component 16 via a pivot pin 44. Thus, the body component 16 is configured to rotate relative to the rod housing 40 along an anterposteriorly extending pivot joint defined by the pivot pin 44. As can be seen in FIG. 8, and as will be described in greater detail below, such rotational movement of the body component 16 relative to the rod housing 40 adjusts the varus/valgus angle of the cutting slot 114 of the distal femoral cutting block 112 when the block 112 is secured to the alignment guide 12 via the outrigger component 14.

The rod housing 40 of the alignment guide 12 has an elongated bore 46 extending therethrough. The elongated bore 46 is configured to receive an intramedullary rod instrument 150 which, in use, is advanced through the bore 46 and into a reamed intramedullary canal 124 of the patient's femur 120. Doing so couples the alignment guide 12 to the distal end 126 of the patient's femur 120 and maintains the alignment of the longitudinal axis 48 of the rod housing's bore 46 with the patient's intramedullary canal 124. When the intramedullary rod instrument 150 is used in such a manner, the rod housing 40 is held in rigid alignment with the center of the reamed intramedullary canal 124 of the patient's femur 120 thus allowing the body component 16 (and hence the distal femoral cutting block 112 secured thereto via the outrigger component 14) to rotate relative to the center of the reamed intramedullary canal 124 of the patient's femur 120.

The alignment guide 12 of the distal femoral jig 10 also includes a locking collar 50 and an end cap 52. The end cap 52 may also be referred to as a cap component 52. The locking collar 50 includes a locking tab 54 distally extending away from its distal edge. The end cap 52 is fixed in position on the proximal end 56 of the rod housing 40. As shown in FIGS. 1-3, the locking collar 50 is slidably positioned on the proximal end 56 of the rod housing 40 at a location between the indexing plate 26 of the body component 16 and the end cap 52. As such, the locking collar 50 may slide back and forth along the rod housing 40 between the indexing plate 26 and the end cap 52. In such a way, the locking collar 50 is positionable between (i) a locked position in which the locking collar 50 is spaced distally apart from the end cap 52 such that the locking tab 54 is positioned in one of the locking notches 30 of the indexing plate 26 so as to lock the rotational position of the rod housing 40 relative to the body component 16, and (ii) a released position in which the locking collar 50 abuts the end cap 52 such that the locking tab 54 is spaced apart from the locking notches 30 of the indexing plate 26 so as to allow the body component 16 to freely rotate relative to the rod housing 40. As will be described in more detail below, such selective positioning of the locking collar 50 allows the distal femoral jig 10 to be operated in either a mechanical alignment mode of operation or a kinematic alignment mode of operation.

As shown in FIG. 4, a spring 60 is positioned on the proximal end 56 of the rod housing 40. The spring 60 biases the locking collar 50 distally toward the indexing plate 26 and into its locked position in which the locking tab 54 is positioned in one of the locking notches 30 of the indexing plate 26 so as to lock the rotational position of the rod housing 40 relative to the body component 16. As can be seen in FIGS. 1-3, both the locking collar 50 and the end cap 52 have a downwardly extending grip 62 formed therein. A surgeon may pinch the two grips 62 toward one another to overcome the bias of the spring 60 thereby moving the locking collar 50 proximally away from the indexing plate 26 and into its released position in which the locking tab 54 is spaced apart from the locking notches 30 of the indexing plate 26 so as to allow the body component 16 to freely rotate relative to the rod housing 40.

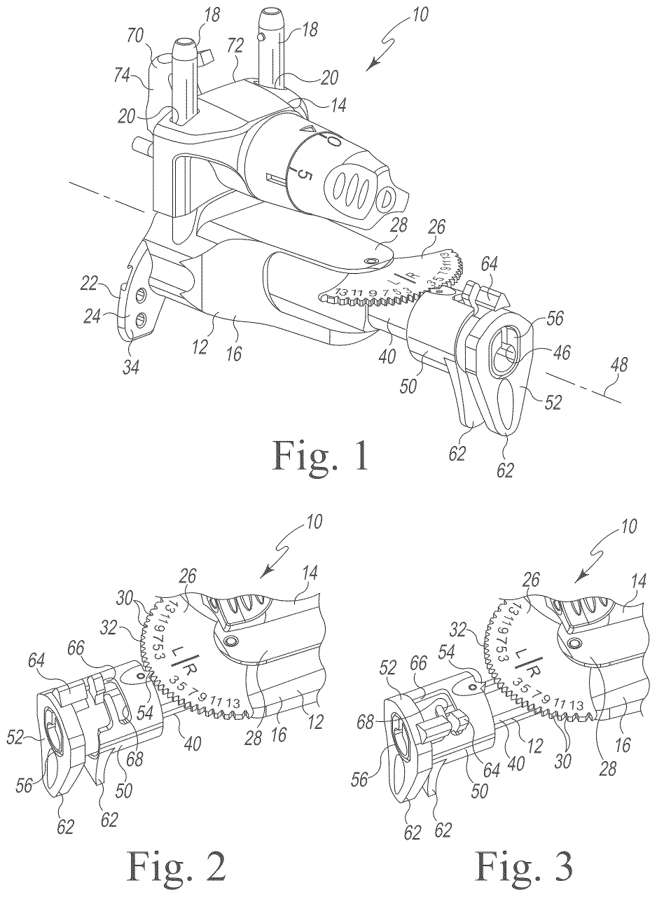

As shown best in FIGS. 2 and 3, the alignment guide 12 of the distal femoral jig 10 includes a retaining lever 64 rotatably positioned on the proximal end 56 of the rod housing 40. The locking collar 50 has a pair of slots 66, 68 formed therein. The slot 66 extends generally parallel to the longitudinal axis 48 of the rod housing's bore 46, whereas the slot 68 extends orthogonally to the longitudinal axis 48 of the rod housing's bore 46. The retaining lever 64 is positionable between a retained position in which the retaining lever 64 retains the locking collar 50 in its released position, and an unretained position in which the locking collar 50 is positioned in its locked position. Specifically, as shown in FIG. 2, when the retaining lever 64 is positioned in its unretained position in which the lever 64 is positioned in the slot 66, the locking collar 50 is free to translate along the rod housing 40. When the retaining lever 64 is so positioned, the spring 60 urges the locking collar 50 into its locked position in which the locking tab 54 is positioned in one of the locking notches 30 of the indexing plate 26 so as to lock the rotational position of the rod housing 40 relative to the body component 16 (unless the bias of the spring 60 is overcome by use of the grips 62). However, as shown in FIG. 3, when the retaining lever 64 is positioned in its retained position in which the lever 64 is positioned in the slot 68, the retaining lever 64 retains the locking collar 50 in its released position in which the locking tab 54 is spaced apart from the locking notches 30 of the indexing plate 26 so as to allow the body component 16 to freely rotate relative to the rod housing 40.

As shown in FIGS. 1 and 8, the outrigger component 14 has a block connector 70 on its distal end 72. In the illustrative embodiment described herein, the block connector 70 is shown as a mounting clip 74, although other types of connectors are contemplated for use. The distal femoral cutting block 112 is clipped or otherwise secured to the outrigger component 14 with the mounting clip 74. As can be seen in FIG. 8, when the distal femoral cutting block 112 is secured to the outrigger component 14 and the outrigger component 14 is assembled to the alignment guide 12, the longitudinal axis 48 of the rod housing's bore 46 and the longitudinal axis 116 of the cutting slot 114 of the distal femoral cutting block 112 define a varus/valgus cutting angle 118. As the alignment guide 12 is rotated relative to the rod housing 40, the outrigger component 14 is likewise rotated relative to the rod housing 40 thereby changing the orientation of the longitudinal axis 116 of the cutting slot 114 of the distal femoral cutting block 112 relative to the longitudinal axis 48 of the rod housing's bore 46 and thus changing the varus/valgus cutting angle 118.

As described above, the surgeon may use the distal femoral jig 10 to adjust the position of the cutting slot 114 of the distal femoral cutting block 112 relative to the patient's femur 120 so as to adjust the block's varus/valgus cutting angle 118 (and hence the varus/valgus angle in which a femoral prosthetic component is implanted). The distal femoral jig 10 may be operated in two different modes of operation based on a given surgeon's preferences. In particular, for a surgeon with a preference for mechanical alignment techniques, the distal femoral jig 10 may be operated in a mechanical alignment mode of operation. Typically, a surgeon utilizing mechanical alignment techniques will prefer to set the varus/valgus cutting angle 118 at a predetermined angle (e.g., 5-7 degrees) relative to the anatomic axis of the femur 120 based on preoperative planning. Thus, the surgeon may operate the distal femoral jig 10 in the mechanical mode of operation by positioning the locking collar 50 in its locked position in which the collar's locking tab 54 is positioned in one of the locking notches 30 of the indexing plate 26 corresponding to the desired varus/valgus cutting angle 118 (left or right—0 degrees to 14 degrees) so as to lock the rotational position of the rod housing 40 relative to the body component 16 in a position that positions the cutting slot 114 of the distal femoral cutting block 112 in the desired varus/valgus cutting angle 118 (left or right—0 degrees to 14 degrees). Alternatively, for a surgeon with a preference for kinematic alignment techniques, the distal femoral jig 10 may be operated in a kinematic alignment mode of operation. Typically, a surgeon utilizing kinematic alignment techniques will prefer to intraoperatively set the varus/valgus cutting angle 118 by positioning the bone facing surface 22 formed in the distal end 24 of the body component 16 in a desired orientation relative to the patient's femur 120. This is generally done by orientating the bone facing surface 22 such that the condylar flanges 34 located on each side of the bone facing surface 22 contacts the distal condyles 128 of the patient's femur 120—i.e., the condylar flange 34 located on the medial side of the bone facing surface 22 contacts the medial distal condyle 128 and the condylar flange 34 located on the lateral side of the bone facing surface 22 contacts the lateral distal condyle 128. To do this, the surgeon typically “floats” the position of the distal femoral cutting block 112, as opposed to selecting from specific predetermined positions as is the case with mechanical alignment techniques. As a result, the surgeon may operate the distal femoral jig 10 in the kinematic mode of operation by positioning the locking collar 50 in its released position in which the collar's locking tab 54 is spaced apart from the locking notches 30 of the indexing plate 26 so as to allow the rod housing 40 to freely rotate (e.g., “float”) relative to the body component 16 to allow the surgeon to position the bone facing surface 22 in a desired position relative to the distal condyles 128 of the patient's femur 120 thereby positioning the cutting slot 114 of the distal femoral cutting block 112 in the desired varus/valgus cutting angle 118.

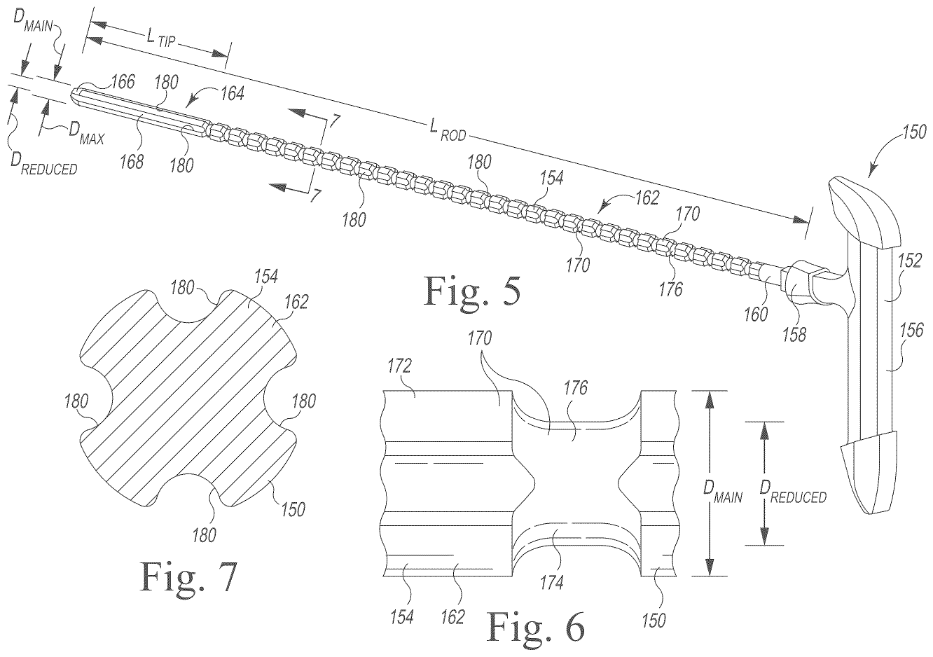

Referring now to FIGS. 5 and 6, there is shown the intramedullary rod instrument 150 in more detail. As alluded to above, in use, the intramedullary rod instrument 150 is advanced through the distal femoral jig's rod housing 40 and into the reamed intramedullary canal 124 of the patient's femur 120 so as to position the distal femoral jig 10 in a desired alignment relative to the anatomic axis of the patient's femur 120. As will be described below in more detail, the intramedullary rod instrument 150 is segmented so as to be “semi-rigid” thereby allowing the rod instrument 150 to flex and thus navigate to the curvatures present in the intramedullary canal 124 of some patients. As such, the intramedullary rod instrument 150 may be “fully seated” within the femur 120 of most patients, including those patients having a curvature of the femur 120 that might otherwise restrict the seating of a fully rigid rod.

The intramedullary rod instrument 150 includes a T-shaped handle 152 and an elongated fluted rod 154. The T-shaped handle 152 includes an anterposteriorly extending grip 156 that has a collar 158 superiorly extending therefrom. A proximal end 160 of the fluted rod 154 is secured to the collar 158 such that the fluted rod 154 extends distally away from the handle 152. In an illustrated embodiment, the handle 152 may be constructed of either a metallic or polymeric material, with the fluted rod 154 being a monolithic component constructed of metal.

The fluted rod 154 includes a segmented proximal body 162 and a non-segmented distal tip 164. As can be seen in FIG. 5, the non-segmented distal tip 164 has a bullet-shaped terminal end 166 and a rod section 168 extending proximally from the terminal end 166. The rod section 168 of the non-segmented distal tip 164 has a constant diameter and, given that it is larger than the diameter of the terminal end 166, defines the maximum diameter (D_{MAX}) of the distal tip 164.

Unlike the non-segmented distal tip 164, the segmented proximal body 162 of the fluted rod 154 has a plurality of shaft segments 170 formed therein. Each of the shaft segments 170 includes a main shaft section 172 and a reduced shaft section 174. As can be seen in FIG. 5, each of the reduced shaft sections 174 is secured to a main shaft section 172 of an adjacent shaft segment 170.

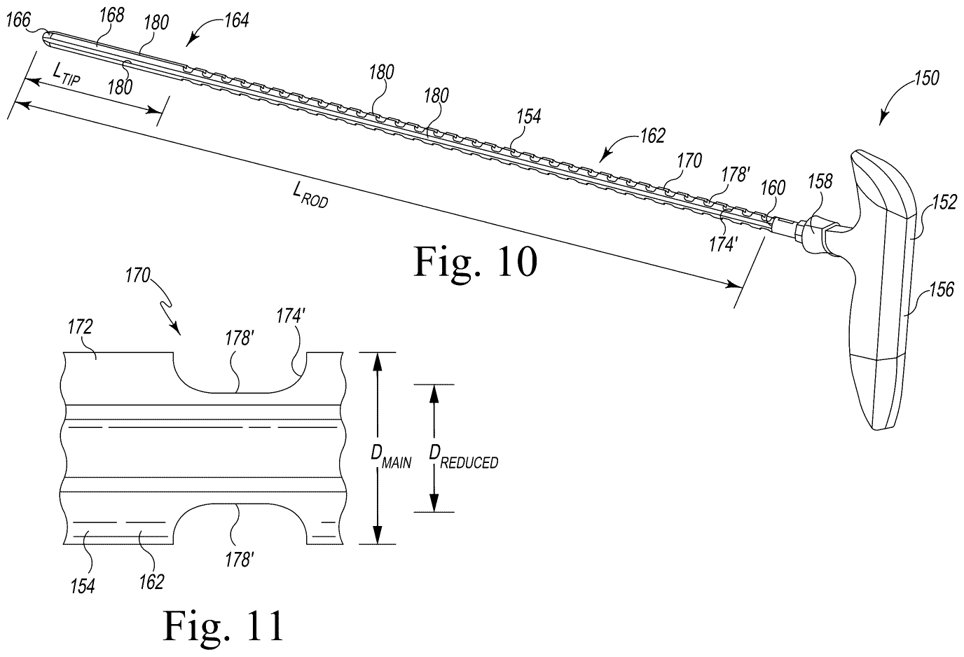

As can be seen in FIGS. 5 and 6, the main shaft section 172 of each shaft segment 170 has a diameter (D_{MAIN}) that is the same as the maximum diameter (D_{MAX}) of the non-segmented distal tip 164 (i.e., D_{MAIN}=D_{MAX}). In some embodiments, as can be seen best in FIG. 6, the reduced shaft section 174 of each shaft segment 170 defines a complete, uninterrupted cylindrical surface 176. In other words, the reduced shaft section 174 defines a reduced surface 176 on each of the anterior, posterior, medial, and lateral sides of the fluted rod 154. In some embodiments, as shown in FIGS. 10 and 11, a reduced shaft section 174′ of each shaft segment 170 defines a reduced surface 178′ on an anterior side and a posterior side of the fluted rod 154. In this way, the reduced shaft section 174′ does not define a reduced surface on a medial side or a lateral side of the fluted rod 154.

Moreover, the reduced shaft section 174, 174′ of each shaft segment 170 has a diameter (D_{REDUCED}) that is less than the maximum diameter (D_{MAX}) of the non-segmented distal tip 164 and the diameter (D_{MAIN}) of the main shaft sections 172 of the rod's segmented proximal body 162. For the reduced shaft section 174′, the diameter (D_{REDUCED}) is measured between the two reduced surfaces 178′, as shown in FIG. 11. In an illustrative embodiment, the diameter (D_{REDUCED}) of the reduced shaft section 174, 174′ of each shaft segment 170 is between 25-75% the size of the maximum diameter (D_{MAX}) of the non-segmented distal tip 164 and the diameter (D_{MAIN}) of the main shaft sections 172 of the rod's segmented proximal body 162. In a more specific illustrative embodiment, the diameter (D_{REDUCED}) of the reduced shaft section 174, 174′ of each shaft segment 170 is approximately 66.6% the size of the maximum diameter (D_{MAX}) of the non-segmented distal tip 164 and the diameter (D_{MAIN}) of the main shaft sections 172 of the rod's segmented proximal body 162.

As can be seen in FIGS. 5 and 10, in an illustrative embodiment, the non-segmented distal tip 164 has a length (L_{TIP}) that is between 20-25% of the overall length (L_{ROD}) of the fluted rod 154. In a more specific embodiment, the non-segmented distal tip 164 has a length (L_{TIP}) that is approximately 20% of the overall length (L_{ROD}) of the fluted rod 154. The non-segmented distal tip 164 provides for a rigid lead in of the fluted rod 154 as the fluted rod 154 is inserted into the intramedullary canal 124.

As can be seen in FIGS. 5 and 7, a number of superinferiorly extending fluid channels 180 are formed in the fluted rod 154. The fluid channels 180 function to permit the channeling of fluids within the intramedullary canal 124 of the patient's femur 120 during use of the intramedullary rod instrument 150. In an embodiment, one of the fluid channels 180 is formed in each of the anterior, posterior, medial, and lateral sides of the fluted rod 154. The fluid channels 180 extend linearly through both the non-segmented distal tip 164 and the segmented proximal body 162 of the fluted rod 154. Thus, the sections of the fluid channels 180 formed in the anterior, posterior, medial, and lateral sides of the non-segmented distal tip 164 are colinear with the sections of the fluid channels 180 formed in the anterior, posterior, medial, and lateral sides of the main shaft section 172 of each of the shaft segments 170 of the segmented proximal body 162, respectively. In the embodiment of FIGS. 10 and 11, the fluid channels 180 are formed in the medial and lateral sides of the reduced shaft section 174′.

In operation, the surgeon may utilize the distal femoral jig 10 and the intramedullary rod instrument 150 during performance of an orthopaedic knee procedure to prepare the distal end 126 of the patient's femur 120 to receive a prosthetic femoral component. To do so, the surgeon may utilize the distal femoral jig 10 to position and install (e.g., pin) the distal femoral cutting block 112 on the patient's femur 120 and thereafter use the installed cutting block 112 to guide a bone saw blade 130 in making a distal cut on the distal end 126 of the patient's femur 120.

As shown in FIG. 8, during such an orthopaedic surgical procedure, the surgeon first orientates the patient's femur 120 such that the patient's knee is positioned in flexion. With the patient's knee positioned in flexion, the surgeon may use a step drill (not shown) or similar instrument to ream or otherwise drill the distal end 126 of the patient's femur 120 to gain access to the intramedullary canal 124. Thereafter, the surgeon assembles a surgical instrument construct. Specifically, the surgeon couples the outrigger component 14 to the alignment guide 12 and thereafter clips the distal femoral cutting block 112 to the outrigger component 14 with the mounting clip 74. Thereafter, the surgeon advances the non-segmented distal tip 164 of the intramedullary rod instrument 150 into the proximal end of the alignment guide's elongated bore 46.

The surgeon then advances the non-segmented distal tip 164 of the intramedullary rod instrument 150 through the alignment guide's elongated bore 46 and out the distal end of the bore 46. Thereafter, the non-segmented distal tip 164 of the intramedullary rod instrument 150 is advanced into the reamed intramedullary canal 124 of the patient's femur 120. As the intramedullary rod instrument 150 is advanced further into the intramedullary canal 124, the segmented proximal body 162 of its fluted rod 154 flexes within bowed sections of the intramedullary canal 124 thereby allowing the rod instrument 150 to be fully seated (i.e., the distal end of the handle's collar 158 abuts the distal femoral jig 10). Such installation of the intramedullary rod instrument 150 into the reamed intramedullary canal 124 of the patient's femur 120 couples the distal femoral jig 10 to the distal end 126 of the patient's femur 120. Such installation of the intramedullary rod instrument 150 into the reamed intramedullary canal 124 of the patient's femur 120 also aligns the rod housing 40 of the distal femoral jig 10 with the anatomic axis of the patient's femur 120.

Once the distal femoral jig 10 has been coupled to the patient's femur 120, the surgeon uses the distal femoral jig 10 to adjust the position of the cutting slot 114 of the distal femoral cutting block 112 relative to the patient's femur 120 so as to adjust the block's varus/valgus cutting angle 118 (and hence the varus/valgus angle in which a femoral prosthetic component is implanted). As noted above, the distal femoral jig 10 may be operated in two different modes of operation based on the surgeon's preferences. If the surgeon has a preference for mechanical alignment techniques, the surgeon selects to operate the distal femoral jig 10 in its mechanical alignment mode of operation. In such a case, the surgeon sets the varus/valgus cutting angle 118 at a predetermined angle (e.g., 5-7 degrees) relative to the anatomic axis of the femur 120 based on preoperative planning. Thus, the surgeon operates the distal femoral jig 10 in the mechanical mode of operation by positioning the locking collar 50 in its locked position in which the collar's locking tab 54 is positioned in the locking notch 30 of the indexing plate 26 corresponding to the desired varus/valgus cutting angle 118 (left or right—0 degrees to 14 degrees) so as to lock the rotational position of the rod housing 40 relative to the body component 16 in a position that orients the cutting slot 114 of the distal femoral cutting block 112 in the desired varus/valgus cutting angle 118 (left or right—0 degrees to 14 degrees).

Alternatively, if the surgeon has a preference for kinematic alignment techniques, the surgeon operates the distal femoral jig 10 in its kinematic alignment mode of operation. In such a case, the surgeon intraoperatively sets the varus/valgus cutting angle 118 by positioning the bone facing surface 22 formed in the distal end 24 of the body component 16 in a desired orientation relative to the patient's femur 120. To do this, the surgeon orients the bone facing surface 22 such that the condylar flanges 34 located on each side of the bone facing surface 22 contacts the distal condyles 128 of the patient's femur 120—i.e., the condylar flange 34 located on the medial side of the bone facing surface 22 contacts the medial distal condyle 128 and the condylar flange 34 located on the lateral side of the bone facing surface 22 contacts the lateral distal condyle 128. While doing this, the surgeon “floats” the position of the distal femoral cutting block 112 by positioning the locking collar 50 in its released position in which the collar's locking tab 54 is spaced apart from the locking notches 30 of the indexing plate 26 so as to allow the rod housing 40 to freely rotate (e.g., “float”) relative to the body component 16 to allow the surgeon to position the bone facing surface 22 in a desired position relative to the distal condyles 128 of the patient's femur 120 thereby positioning the cutting slot 114 of the distal femoral cutting block 112 in the desired varus/valgus cutting angle 118. During such free movement of the distal femoral cutting block 112, the surgeon may pinch the grips 62 on the locking collar 50 and the end cap 52 to maintain the locking collar 50 in its released position, or, alternatively, utilize the retaining lever 64 to retain the locking collar 50 in its released position.

Whether by use in its mechanical alignment mode of operation or its kinematic alignment mode of operation, once the surgeon has positioned the cutting slot 114 of the distal femoral cutting block 112 in the desired varus/valgus cutting angle 118, the surgeon may pin the distal femoral cutting block 112 to the distal end 126 of the patient's femur 120 by installing a pair of bone pins 132 through a selected pair of the block's pin holes. With the distal femoral cutting block 112 installed on the distal end 126 of the patient's femur 120, the surgeon removes the distal femoral jig 10 by unclipping the cutting block 112 from the outrigger component 14 and thereafter pulling the T-shaped handle 152 of the intramedullary rod instrument 150 and the distal femoral jig 10 in a direction away from the patient's femur 120 until the rod instrument 150 exits from the intramedullary canal 124 of the patient's femur 120 thereby leaving the pinned cutting block 112, as shown in FIG. 9. As also shown in FIG. 9, the surgeon may then use the distal femoral cutting block 112 to perform a distal resection of the distal end 126 of the patient's femur 120. Specifically, the surgeon may advance the bone saw blade 130 of a surgical saw through the distal cutting slot 114 to engage the patient's femur 120 and operate the surgical saw to surgically form a planar distal resected surface of the patient's femur 120. The surgeon may then pin an additional cutting block, such as a 4-in-1 cutting block (not shown) on the surgically-prepared distal resected surface and thereafter perform additional resections such as an anterior cut, a posterior cut, and a pair of chamfer cuts. Once the anterior cut, posterior cut, and both chamfer cuts have been made, the surgeon removes the 4-in-1 cutting block from the patient's femur 120 and completes the orthopaedic knee procedure.

It should be appreciated that use of the distal femoral jig 10 and the intramedullary rod instrument 150 in combination has been described herein and such a combination has significant advantages. However, certain of such advantages may be achieved by use of the two instruments separate from one another. For example, the distal femoral jig 10 may be used with a traditional (e.g., non-segmented) intramedullary rod instrument. Similarly, the segmented intramedullary rod instrument 150 may be used with traditional distal femoral jigs.

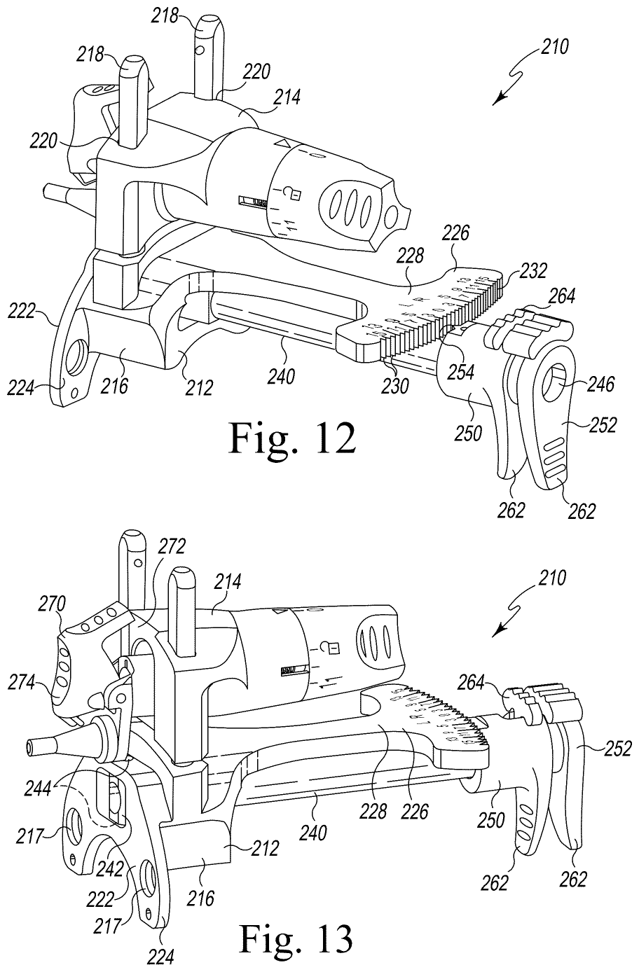

Referring to FIGS. 12 and 13, in another embodiment, an orthopaedic surgical instrument 210—in the form of a distal femoral jig—for use in the surgical preparation of a patient's femur during performance of an orthopaedic knee procedure is shown. The distal femoral jig 210 of FIGS. 12 and 13 is substantially similar to the distal femoral jig 10 shown in FIGS. 1-4 and 8 and described herein. Accordingly, similar reference numbers are used in the description of the distal femoral jig 210 to indicate features that are common between the distal femoral jig 10 and the distal femoral jig 210. The description of the distal femoral jig 10 is incorporated by reference to apply to the distal femoral jig 210, except in instances when it conflicts with the specific description and the drawings of the distal femoral jig 210.

The distal femoral jig 210 includes an alignment guide 212 and a removable outrigger component 214. As can be seen in FIGS. 12 and 13, the alignment guide 212 of the distal femoral jig 210 includes a hollow body component 216 having a pair of anteriorly extending mounting posts 218. The outrigger component 214 includes a pair of mounting bores 220 which receive the mounting posts 218 to removably secure the outrigger component 214 to the body component 216. The body component 216 has a bone facing surface 222 formed in its distal end 224.

As shown in FIG. 13, the body component 216 is formed to include two holes 217 on opposing medial and lateral sides of the distal end 224. The two holes 217 extend entirely superoinferiorly through the distal end 224. The two holes 217 receive respective shims (not shown) therein. The shims have varying thicknesses (e.g., 1 mm, 2 mm, 3 mm, etc.) to account for localized cartilage loss. Different shims may be used for both of the two holes 217 to account for different localized cartilage loss on the medial distal condyle 128 and the lateral distal condyle 128.

An indexing plate 226 is secured to a proximal end 228 of the body component 216. The indexing plate 226 has a plurality of locking notches 230 radially positioned around its proximal end 232. Each of the plurality of locking notches 230 represents a desired varus/valgus angle (left or right—0 degrees to 16 degrees) for selection by a surgeon during use of the distal femoral jig 210.

The alignment guide 212 also includes an elongated, hollow rod housing 240 positioned in the hollow body component 216. As can be seen in FIG. 12, a distal end 242 of the rod housing 240 is pivotally coupled to the distal end 224 of the body component 216 via one or more pivot pins 244. Thus, the rod housing 240 is configured to rotate relative to the body component 216 along an anterposteriorly extending pivot joint defined by the one or more pivot pins 244.

The rod housing 240 of the alignment guide 212 has an elongated bore 246 extending therethrough. The elongated bore 246 is configured to receive the intramedullary rod instrument 150.

The alignment guide 212 of the distal femoral jig 210 also includes a locking collar 250 and an end cap 252. The locking collar 250 includes a locking tab 254 extending distally away from its distal edge. The end cap 252 is fixed in position on the proximal end of the rod housing 240. As described above in relation to the locking collar 250, the locking collar 250 is slidably positioned on the proximal end of the rod housing 240 at a location between the indexing plate 226 of the body component 216 and the end cap 252. As such, the locking collar 250 may slide back and forth along the rod housing 240 between the indexing plate 226 and the end cap 252. In such a way, the locking collar 250 is positionable between (i) a locked position in which the locking collar 250 is spaced distally apart from the end cap 252 such that the locking tab 254 is positioned in one of the locking notches 230 of the indexing plate 226 so as to lock the rotational position of the rod housing 240 relative to the body component 216, and (ii) a released position in which the locking collar 250 abuts the end cap 252 such that the locking tab 254 is spaced apart from the locking notches 230 of the indexing plate 226 so as to allow the rod housing 240 to freely rotate relative to the body component 216. The locking collar 250 is slidable along a superoinferiorly extending axis that is perpendicular to the anteroposteriorly extending pivot joint defined by the one or more pivot pins 44 about which the rod housing 240 and/or the body component 216 rotate.

As can be seen in FIGS. 12 and 13, both the locking collar 250 and the end cap 252 have a downwardly extending grip 262 formed therein. The grips 262 may be pinched by the surgeon such that the locking collar 250 is in the released position. The surgeon may then rotate the rod housing 240 relative to the body component 216 (and hence the distal femoral cutting block 112 secured thereto via the outrigger component 214).

The alignment guide 212 of the distal femoral jig 210 includes a retaining lever 264 rotatably positioned on the proximal end of the rod housing 240. The retaining lever 264 is positionable between a retained position in which the retaining lever 264 retains the locking collar 250 in its released position, and an unretained position in which the locking collar 250 is positioned in its locked position. The retaining lever 264 rotates about the superoinferiorly extending axis that the locking collar 250 slides along.

As shown in FIG. 13, the outrigger component 214 has a block connector 270 on its distal end 272. In the illustrative embodiment described herein, the block connector 270 is shown as a mounting clip 274, although other types of connectors are contemplated for use. The distal femoral cutting block 112 is clipped or otherwise secured to the outrigger component 214 with the mounting clip 274.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument assembly for resecting distal condyles of a patient's femur during an orthopaedic knee replacement procedure, comprising:
a distal femoral cutting block having a mediolaterally extending cutting slot,
an outrigger component having a block connector on its distal end, the distal femoral cutting block being secured to the outrigger component with the block connector,
a hollow body component secured to the outrigger component, the body component having (i) a bone facing surface formed in its distal end, the bone facing surface being configured to abut the distal condyles of the patient's femur, and (ii) an indexing plate secured to a proximal end of the body component, the indexing plate having a plurality of locking notches radially positioned around a proximal end thereof, an elongated, hollow rod housing positioned in the body component,
wherein (i) a distal end of the rod housing is pivotally coupled to the distal end of the body component, and (ii) the rod housing has an elongated bore extending therethrough that is configured to receive an intramedullary rod instrument, and
a locking collar slidably positioned on a proximal end of the rod housing, the locking collar having a locking tab distally extending away therefrom, the locking collar being positionable between (i) a locked position in which the locking tab is positioned in one of the locking notches of the indexing plate so as to lock the rotational position of the rod housing relative to the body component, and (ii) a released position in which the locking tab is spaced apart from the locking notches of the indexing plate so as to allow the body component and the rod housing to freely rotate relative to one another.

2. The orthopaedic surgical instrument assembly of claim 1, further comprising a cap component positioned on the proximal end of the rod housing, wherein:
the locking collar is positioned between the indexing plate and the cap component,
the locking collar abuts the cap component when the locking collar is positioned in its released position, and
the locking collar is spaced distally apart from the cap component when the locking collar is positioned in its locked position.

3. The orthopaedic surgical instrument assembly of claim 1, further comprising a retaining lever rotatably positioned on the proximal end of the rod housing, wherein the retaining lever is positionable between (i) a retained position in which the retaining lever retains the locking collar in its released position, and (ii) an unretained position in which the locking collar is positioned in its locked position.

4. The orthopaedic surgical instrument assembly of claim 1, further comprising a spring positioned on the proximal end of the rod housing, the spring biases the locking collar toward the indexing plate.

5. The orthopaedic surgical instrument assembly of claim 1, wherein:
a longitudinal axis of the elongated bore of the rod housing and a longitudinal axis of the cutting slot of the distal femoral cutting block define a cutting angle, and
rotational movement of the body component and the rod housing relative to one another alters the cutting angle.

6. The orthopaedic surgical instrument assembly of claim 5, wherein each of the plurality of locking notches of the indexing plate corresponds to a predetermined cutting angle.

7. The orthopaedic surgical instrument assembly of claim 1, further comprising a first grip coupled to the locking collar and a second grip coupled to the proximal end of the of the rod housing.

8. The orthopaedic surgical instrument assembly of claim 7, wherein the first grip is moveable relative to the second grip to cause the locking collar to move between its locked position and its released position.

9. The orthopaedic surgical instrument assembly of claim 1, wherein the locking collar includes a first slot and a second slot formed therein, the first slot extending generally parallel to a longitudinal axis of the elongated bore of the rod housing and the second slot extending orthogonally to the longitudinal axis of the elongated bore of the rod housing.

10. The orthopaedic surgical instrument assembly of claim 9, further comprising a retaining lever rotatably positioned on the proximal end of the rod housing, wherein the retaining lever is positionable between (i) a retained position in which the retaining lever is positioned in the second slot to retain the locking collar in its released position, and (ii) an unretained position in which the retaining lever is positioned in the first slot to position the locking collar in its locked position.

11. A method of surgically preparing a patient's femur during an orthopaedic surgical knee procedure, comprising:
securing a distal femoral cutting block to a hollow body component of a distal femoral jig,
selecting between a kinematic alignment mode of operation of the distal femoral jig and a mechanical alignment mode of operation of the distal femoral jig,
positioning, if the mechanical alignment mode of operation was selected, a locking collar of the distal femoral jig in a locked position so as to lock the distal femoral cutting block into one of a plurality of predetermined rotational positions, and
positioning, if the kinematic mode of operation was selected, the locking collar of the distal femoral jig in a released position so as to allow the distal femoral cutting block to rotate freely.

12. The method of claim 11, wherein securing the distal femoral cutting block to the hollow body component comprises (i) securing the distal femoral cutting block to an outrigger component of the distal femoral jig, and (ii) securing the outrigger component to the hollow body component.

13. The method of claim 11, wherein:
the distal femoral jig includes a hollow rod housing positioned in, and pivotally coupled to, the hollow body component,
the hollow body component has an indexing plate secured to a proximal end thereof, the indexing plate comprising a plurality of locking notches,
the locking collar is positioned on the rod housing and includes a locking tab,
positioning the locking collar of the distal femoral jig in the locked position comprises positioning the locking tab of the locking collar in one of the plurality of locking notches of the indexing plate so as to lock the rotational position of the rod housing relative to the body component.

14. The method of claim 13, wherein positioning the locking collar of the distal femoral jig in the released position comprises retaining the locking tab of the locking collar spaced apart from the plurality of locking notches of the indexing plate so as to allow the body component and the rod housing to rotate freely relative to one another.

15. The method of claim 14, wherein retaining the locking tab of the locking collar spaced apart from the plurality of locking notches of the indexing plate comprises retaining the locking collar in its retained position with a retaining lever.

16. The method of claim 11, wherein:
the distal femoral jig includes a hollow rod housing positioned in, and pivotally coupled to, the hollow body component, and
the method further comprises positioning the hollow body component of the distal femoral jig such that a bone facing surface of the hollow body component abuts distal condyles of the patient's femur and advancing an intramedullary rod instrument through the hollow rod housing and into a reamed intramedullary canal of the patient's femur.

17. A method of surgically preparing a patient's femur during an orthopaedic surgical knee procedure, comprising:
securing a distal femoral cutting block to a distal femoral jig,
advancing an intramedullary rod instrument through the distal femoral jig,
selecting between a kinematic alignment mode of operation of the distal femoral jig and a mechanical alignment mode of operation of the distal femoral jig,
positioning, if the mechanical alignment mode of operation was selected, a locking collar of the distal femoral jig in a locked position so as to lock the distal femoral cutting block into one of a plurality of predetermined rotational positions, and
positioning, if the kinematic mode of operation was selected, the locking collar of the distal femoral jig in a released position so as to allow the distal femoral cutting block to rotate freely.

18. The method of claim 17, wherein:
the distal femoral jig includes a hollow rod housing positioned in, and pivotally coupled to, a hollow body component,
the hollow body component has an indexing plate secured to a proximal end thereof, the indexing plate comprising a plurality of locking notches,
the locking collar is positioned on the rod housing and includes a locking tab,
positioning the locking collar of the distal femoral jig in the locked position comprises positioning the locking tab of the locking collar in one of the plurality of locking notches of the indexing plate so as to lock the rotational position of the rod housing relative to the body component.

19. The method of claim 18, wherein positioning the locking collar of the distal femoral jig in the released position comprises retaining the locking tab of the locking collar spaced apart from the plurality of locking notches of the indexing plate so as to allow the body component and the rod housing to rotate freely relative to one another.

20. The method of claim 19, wherein retaining the locking tab of the locking collar spaced apart from the plurality of locking notches of the indexing plate comprises retaining the locking collar in its retained position with a retaining lever.
